(19) United States
(12) Patent Application Publication
(10) Pub. No.: US 2023/0022942 A1
(43) Pub. Date: Jan. 26, 2023

(54) METHOD FOR ANALYZING ILEOSTOMY SUBJECTS USING A PROBIOTIC CONTAINING BACILLUS SUBTILIS

(71) Applicant: TEAGASC, The Agriculture and Food Development Authority, Co., Carlow (IE)

(72) Inventors: Martin Joseph Mary Buckley, Co. Cork (IE); Daniela Alexandra da Silva Freitas, Azores (PT); Andre Brodkorb, Co. Cork (IE); John Deaton, Kennesaw, GA (US)

(21) Appl. No.: 17/872,540

(22) Filed: Jul. 25, 2022

Related U.S. Application Data
(60) Provisional application No. 63/225,144, filed on Jul. 23, 2021.

(51) Int. Cl. A23L 33/135

(52) U.S. Cl. CPC A23L 33/135

(57) ABSTRACT

The disclosure relates to a method for a non-invasive technique to analyze the fate of probiotics and commensal microbiota from the human upper gastrointestinal tract, namely the ileum. The invention further relates to methods for the enumeration and determination of mechanism of action of probiotics in a non-invasive manner from the end of the small intestine. A composition comprising a probiotic delivered in vivo to healthy individuals with an ileostomy bag is described.

### CROSS-REFERENCE TO RELATED APPLICATIONS

The present application claims the benefit of priority based on U.S. Provisional Application No. 63/225,144, filed on Jul. 23, 2021. Furthermore, the entire content and disclosure of the US provisional application is incorporated herein by reference in its entirety.

### TECHNICAL FIELD

This disclosure relates to a method for a non-invasive technique to analyze the fate of probiotics and commensal microbiota from the human upper gastrointestinal tract, namely the ileum. The invention further relates to methods for the enumeration and determination of mechanism of action of probiotics in a non-invasive manner from the end of the small intestine. A composition comprising a probiotic delivered in vivo to healthy individuals with an ileostomy bag.

### BACKGROUND

Probiotics are defined as “live microorganisms that, when administered in adequate amounts, confer a health benefit on the host” (Hill C, Guarner F, Reid G, Gibson G. R., Merenstein D. J., Pot B, Morelli L, Canani R. B., Flint H. J., Salminen S, et al. Expert consensus document: The International Scientific Association for Probiotics and Prebiotics consensus statement on the scope and appropriate use of the term probiotic, Nat. Rev. Gastroenterol. Hepatol. 2014. 11: 506-514). Probiotics act through diverse mechanisms that affect the host microbiota. The main mechanisms of action of probiotics are (i) modification of the composition or function of gut microbiota, (ii) improving the immune response and (iii) inhibiting gut pathogens. In order to be successful in these actions, probiotics must survive transit through various stages of the human gastrointestinal tract and typically colonise, even briefly, the intestine.

Typically, enumeration studies on probiotics in human subjects looks at microbial counts, including those of commensal microbes, in stool samples. This however does not accurately reflect the life cycle or actions of the probiotic through the gastrointestinal tract. In particular, the fate of anaerobic/facultative anaerobic microbial probiotics that would have greatest functionality and impact in the oxygen depleted small intestine is missed.

Hence, there is a need for a method to investigate the fate and mechanisms of probiotics in the human small intestine in a non-invasive manner. In one embodiment, an object of this invention is to define a methodology for the investigation of the fate, enumeration and mechanisms of actions of probiotics as well as analysis of commensal microbiota from the small intestine.

### SUMMARY

In view of the current disadvantages of analysis of probiotic fate and actions in the small intestine of humans, in one embodiment the present invention provides a novel, non-invasive technique for analysis of probiotics from the ileum section of the gastrointestinal tract, whereas the same can be used for the analysis of commensal microbial communities, including changes following ingestion of probiotics/prebiotics/synbiotics, of the small intestine.

The general purpose of the invention, which will be described subsequently in greater detail, is to provide a new, non-invasive technique for the analysis of the fate, enumeration and action of ingested probiotics.

According to an embodiment of the invention, ileostomy patients ingest a defined quantity of a specific probiotic/probiotic mix/prebiotic/synbiotic, and the fate, enumeration and mechanisms of actions of the ingested material are analysed through collection and processing of stoma bag contents at defined timepoints following product ingestion.

Accordingly, in an embodiment the invention uniquely makes use of ileostomy bags and contents to analyse the fate, enumeration and mechanisms of actions of consumed probiotics/prebiotics/synbiotics. Additionally, analysis of commensal microbiota is also able to be performed, adding additional crucial information as to changes in the state of the small intestine upon probiotic ingestion.

Other aspects and preferred embodiments of the invention are defined and described in the other claims set out below.

### BRIEF DESCRIPTION OF THE FIGURES

FIG. 1. Flowchart of the study profile.

FIG. 2. Baseline characteristics of the participants.

FIG. 3. B. subtilis DE111® concentration in the small intestinal tract of healthy individuals with an ileostomy stoma. Vegetative DE111® (●), DE111® spores (▪), and placebo (▴). Values are average concentrations (n=11)±standard deviation.

FIG. 4. Bacillus subtilis DE111® relative spore and vegetative cell concentration (% of total DE111® counts) in ileal effluents of individual participants (A-K) over the course of the study session (0-8 h).

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

An ileostomy is a surgical opening that is created by bringing the end or loop of the ileum out to the surface of the skin. Waste from the small intestine passes out of the ileostomy and is collected in a drainable stoma bag which is emptied several times a day and changed every few days.

According to one embodiment of the invention, ileostomy patients ingest a defined quantity of a specific probiotic/probiotic mix/prebiotic/synbiotic, and the fate, enumeration and mechanisms of actions of the ingested material are analysed through collection and processing of stoma bag contents at defined timepoints following product ingestion.

It is further contemplated that one or more beneficial organisms, such as, for example, bacterial species (spp.) may be combined in a probiotic formulation such as those listed above.

As used herein, an “effective amount” or an “amount effective for” is defined as an amount effective, at dosages and for periods of time necessary, to achieve a desired biological result, such as inducing a particular beneficial effect. The effective amount of compositions of the disclosure may vary according to factors such as age, sex, and weight of the individual. Dosage regime may be adjusted to provide the optimum response. Several divided doses may be administered daily, or the dose may be proportionally reduced as indicated by the exigencies of an individual's situation. As will be readily appreciated, a composition in accordance with the present disclosure may be administered in a single serving or in multiple servings spaced throughout the day. As will be understood by those skilled in the art, servings need not be limited to daily administration, and may be on an every second or third day or other convenient effective basis. The administration on a given day may be in a single serving or in multiple servings spaced throughout the day depending on the exigencies of the situation.

In various embodiments, the present disclosure provides probiotic compositions, methods of producing these probiotic compositions, and methods of favorably administering an effective amount of the probiotic compositions to a subject in need thereof.

In embodiments of the present invention, the probiotic compositions may contain a probiotic microorganism that in some applications may be a spore-based probiotic organism selected from the following genuses: Lactobacillus, Bifidobacterium, Lactococcus, Propionibacterium, Bacillus, Enterococcus, Escherichia, Streptococcus, Pediococcus, and Saccharomyce. In certain aspects, the probiotic microorganism is at least one of Lactobacillus acidophilus, Lactobacillus rhamnosus, Lactobacillus fermentum, Lactobacillus casei, Lactobacillus bulgaricus, Lactobacillus gasseri, Lactobacillus helveticus, Lactobacillus johnsonii, Lactobacillus lactis, Lactobacillus plantarum, Lactobacillus reuteri, Lactobacillus salivarius, Lactobacillus paracasei, Bifidobacterium sp., Bifidobacterium longum, Bifidobacterium infantis, Bifidobacterium animalis, Bifidobacterium bifidum, Bifidobacterium adelocentis, Bifidobacterium lactis, Bacillus subtilis, Bacillus coagulans, Bacillus licheniformis, Enterococcus faecalis, Enterococcus faecium, Lactococcus lactis, Streptococcus salivarius, Saccharomyces cerevisiae, and Saccharomyces boulardii. The probiotic microorganism may be in the form of spores or in a vegetative state.

The Lactobacillus genus is extremely diverse and expanding every year. With over 230 species, it has grown into one of the biggest genera in the bacterial taxonomy. As the genus has exceeded the acceptable “normal diversity,” renaming and re-classification is inevitable wherein the genus Lactobacillus may be split into most likely twelve new genera. Many traditional “probiotic” species with substantiated industrial importance and starter cultures many no longer eventually be called “Lactobacillus.” Hence, a substantial communication challenge looms ahead to reduce the inevitable confusion regarding the “old commercial” and “correct scientific” nomenclature. Once the International Committee on Systematics of Prokaryotes publishes new nomenclature in their official journal, the INTERNATIONAL JOURNAL OF SYSTEMATIC AND EVOLUTIONARY MICROBIOLOGY, the changes are valid and official. The manuscript that will be submitted for publication outlining the new nomenclature of the Lactobacillus genus will likely be ready for submission by the end of 2018. Meanwhile, there was a taxonomic subcommittee meeting in September 2018 to discuss the nomenclature changes and an (invite-only) expert LABIP workshop in October 2018 that will evaluate the science while considering the consequences for regulations, legal/IP, and industry.

Probiotics are measured by colony forming units (“CFUs”). Few studies have been done to determine effective dosages, but effective dosages may be in the millions of CFUs, and are usually in the hundreds of millions of CFUs or higher. If probiotics are being used to help with digestion, probiotics should be taken with meals, but otherwise the probiotics may survive better if taken between meals, particularly if taken with liquids that help to dilute stomach acid and move the probiotics more quickly into the digestive tract. Probiotics may be given short-term or long-term.

In certain embodiments, the concentration of the probiotic microorganism (or combination of probiotic organisms or species) in the composition may be at least about 1×·10⁹ CFU/g, at least about 2×·10⁹ CFU/g, at least about 3×·10⁹ CFU/g, at least about 4×·10⁹ CFU/g, at least about 5×·10⁹ CFU/g, at least about 6×·10⁹ CFU/g, at least about 7×·10⁹ CFU/g, at least about 8×·10⁹ CFU/g, at least about 9×·10⁹ CFU/g, at least about 1×·10¹⁰ CFU/g, at least about 2×·10¹⁰ CFU/g, at least about 3×·10¹⁰ CFU/g, at least about 4×·10¹⁰ CFU/g, at least about 5×·10¹⁰ CFU/g, at least about 6×·10¹⁰ CFU/g, at least about 7×·10¹⁰ CFU/g, at least about 8×·10¹⁰ CFU/g, at least about 9×·10¹⁰ CFU/g, or at least about 1×·10¹¹ CFU/g.

One useful Bacillus subtilis-containing composition is DE111® (“DE111”), available from Deerland Enzymes, Inc. (Kennesaw, Ga., United States). DE111 is an isolated strain of Bacillus subtilis subspecies inaquosorum having accession number NRRL B-67989. The Bacillus subtilis subspecies inaquosorum (DE111) strain was deposited with the Agricultural Research Service Culture Collection (NRRL), an International Depositary Authority, 1815 N. University Street, Peoria, Ill., 61604, United States, under the Budapest Treaty on the International Recognition of the Deposit of Microorganisms, on Sep. 28, 2020 and was accepted and found to be viable on Sep. 28, 2020, and assigned accession number NRRL B-67989. The DE111 strain is a biologically pure culture prepared by a proprietary process.

The Bacillus subtilis DE111 strain has certain properties, which, surprisingly, have been found to make the strain well-suited for use as a probiotic. Spores of Bacillus subtilis are viable under a wide temperature and pH range. Without being bound by any particular theory, it is thought that the ability of Bacillus subtilis DE111 to form spores that protect the microbes from harsh conditions until they enter an environment ripe for germination, such as the GI tract, makes Bacillus particularly well-suited for use as a probiotic.

In one aspect of the invention, compositions administered to patients in need thereof according to the methods of the present disclosure comprise mutants of Bacillus subtilis DE111 having all the identifying characteristics of Bacillus subtilis DE111. Such mutants may have DNA sequence identity to Bacillus subtilis DE111 of at least about 90%, at least about 91%, at least about 92%, at least about 93%, at least about 94%, at least about 95%, at least about 96%, at least about 97%, at least about 98%, or at least about 99%. In some embodiments, mutants are spontaneous mutants. The teen spontaneous mutant refers to mutants that arise from Bacillus subtilis DE111 without the intentional use of mutagens. Such spontaneous mutants may be obtained by classical methods, such as growing the Bacillus subtilis DE111 strain in the presence of a certain antibiotic to which the parent is susceptible, and testing any resistant mutants for improved biological activity or, in this application, improved ability to reduce the symptoms of gastrointestinal irregularity. Other methods for identifying spontaneous mutants will be known to those of ordinary skill in the art.

All references in this application to Bacillus subtilis DE111 or its mutants refer to bacteria that have been isolated from nature and are grown by humans, for example, in the laboratory or under industrial conditions.

Bacillus subtilis DE111 cells may be present in the compositions administered to patients in need thereof according to the methods of the present disclosure as spores (which are dormant), as vegetative cells (which are growing), as transition state cells (which are transitioning from growth phase to sporulation phase) or as a combination of all of these types of cells. In some embodiments, the composition comprises mainly spores. In other embodiments, the composition comprises spores and metabolites produced by the cells during fermentation before they sporulate, as described below.

Compositions administered to patients in need thereof according to the methods of the present disclosure can be obtained by culturing Bacillus subtilis DE111 or its mutants according to methods well known in the art. Conventional large-scale microbial culture processes include submerged fermentation, solid state fermentation, or liquid surface culture. Towards the end of fermentation, as nutrients are depleted, Bacillus subtilis DE111 cells begin the transition from growth phase to sporulation phase, such that the final product of fermentation is largely spores, metabolites, and residual fermentation medium. Sporulation is part of the natural life cycle of Bacillus subtilis DE111 and is generally initiated by the cell in response to nutrient limitation. Fermentation is configured to obtain high levels of colony forming units of Bacillus subtilis DE111 and to promote sporulation. The bacterial cells, spores, and metabolites in culture media resulting from fermentation may be used directly or concentrated by conventional industrial methods, such as centrifugation, tangential-flow filtration, depth filtration, and evaporation. In some embodiments, the concentrated fermentation broth is washed, for example, via a diafiltration process, to remove residual fermentation broth and metabolites.

The fermentation broth or broth concentrate can be dried with or without the addition of carriers using conventional drying processes or methods such as spray drying, freeze drying, tray drying, fluidized-bed drying, drum drying, or evaporation. The resulting dry products may be further processed, such as by milling or granulation, to achieve a specific particle size or physical format. Carriers, described below, may also be added post-drying.

In embodiments in which compositions formulated separately from food or drink are administered to patients in need thereof according to the methods of the present disclosure, the concentration on a weight by weight basis (w/w) of (i) Bacillus subtilis DE111 or its mutants, (ii) metabolites of Bacillus subtilis DE111 or its mutants, or (iii) combinations of cells and metabolites in the formulated composition may be about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, about 90%, or about 95%. In some embodiments of compositions administered to patients in need thereof according to the methods of the present disclosure, where the concentrated formulation broth has been washed and dried without heat, such as via freeze drying, the concentration of Bacillus subtilis DE111 or its mutants in the final composition may be from about 90% to about 100%.

### Exemplification

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### Example 1

A. Sampling

A single bacterial probiotic strain (CFU=1×10⁹) is administered with food to subjects who wear a stoma bag due to having an ileostomy. Stoma bags may be collected at different time points from 0 to 24 hours (e.g. 0 hr, 3 hr, 6 hr, 10 hr, 24 hr) following ingestion of the probiotic. Contents of the stoma bags are emptied into sterile specimen bags and immediately weighed. An aliquot from each sample is taken and blended under sterile conditions. Aliquots from each of the blended samples were taken for dry weight analysis, enumeration and microbiome analysis and stored appropriately prior to analysis.

In an embodiment, the bacterial strain is an isolated strain of Bacillus subtilis subspecies inaquosorum having accession number NRRL B-67989.

B. Dry Weight Analysis

C. Enumeration

D. Microbiome Analysis

The analytical techniques performed in steps B, C, and D are well known in the art.

It is expected that this method to investigate the fate and mechanisms of probiotics in the human small intestine in a non-invasive manner will be effective to elucidate said fate and mechanism and to demonstrate key metrics susceptible to measurement.

It is further expected that ileostomy patients who ingest a defined quantity of a specific probiotic/probiotic mix/prebiotic/symbiotic composition, will benefit after measurement and analysis of the fate, enumeration and mechanisms of actions of the ingested composition.

It is further expected that ileostomy patients who ingest an effective amount or quantity of a specific probiotic/probiotic mix/prebiotic/symbiotic composition, will benefit after measurement and analysis of the fate, enumeration and mechanisms of actions of the ingested composition.

### Example 2— Clinical Study

Materials And Methods:

Study Design

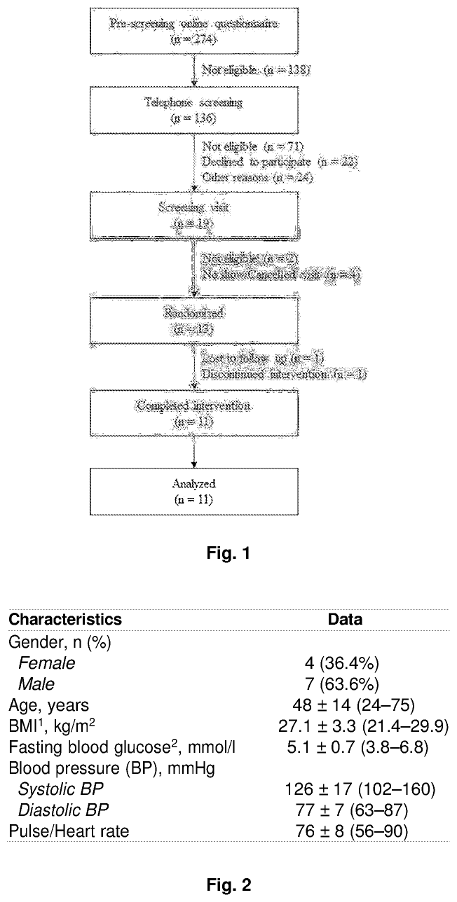

The current study was performed on ileal effluent samples obtained from a wider 4-arm study evaluating the impact of meal properties and dietary supplementation on digestion. This randomized, crossover, double-blind, and placebo-controlled study was carried out between October 2020 and April 2021 at a single site in Ireland. Each of the four interventions was composed of one meal and one study product (placebo or active treatment). The results presented here correspond to two arms only: (1) Meal A+placebo; (2) Meal A+probiotic strain Bacillus subtilis DE111. A flowchart of the study is depicted in FIG. 1. The protocol was approved by the Clinical Research Ethics Committee of the Cork Teaching Hospitals (review reference number: ECM 4 (d) 05/05/2020) and registered on clinicaltrials.gov (NCT04489810). Adults (aged 18-75) were recruited on the basis of inclusion (having an ileostomy stable for at least 3 months post-operation showing normal stoma function and were otherwise healthy) and exclusion (obstruction of the stoma in the previous 3 months, body mass index below 18 kg/m² or above 30 kg/m², being immunocompromised, history of bariatric surgery, history of drug and/or alcohol abuse, and concurrent participation in other research studies, not using an acceptable method of contraception and not pregnant) criteria. In addition, a current or past diagnosis of one or more of the following was also an exclusion criterion: coeliac disease, allergy to wheat and/or any other ingredients in the study meals; mouth, throat, or active gastrointestinal pathology (other than ileostomy) that may affect normal ingestion and digestion of food; pancreatic disease; and diabetes (both Type 1 and Type 2). Participants were asked not to use any proton pump inhibitors or anti-diarrheal medication during the week and day, respectively, preceding each study day. They were also asked to refrain from excessive alcohol consumption and intensive physical activity the day before the study sessions. All participants gave their written informed consent to participate after receiving oral and written information about the research and study protocol. The study was conducted following the principles of the WMA Declaration of Helsinki and ICH-Good Clinical Practice guidelines. Eleven participants received either B. subtilis DE111® (5×10⁹ CFU) or placebo in the morning at breakfast. Each participant received both interventions in this study, in a random sequence and on different days scheduled at least 1 week apart. Participants consumed a standard gluten-free dinner no later than 21 h00 the evening before each study session. Only water was allowed after dinner. The following morning, participants arrived at the study site in a fasted state and remained on-site for the duration of the study session. Participants emptied their ileostomy pouch into a sample collection bag prior to consumption of the investigational products and standard breakfast [two pots of porridge (Flahavan's Organic Original Porridge), one Weetabix, and one glass of water (125 ml), consisting of 400 g of a smooth coup (Cully and Sully, Cork, Ireland) and 150 g of jelly (Boyne Valley Group, Louth Ireland) was consumed by all participants. Throughout the study session, participants' water intake was monitored but unrestricted (up to 1.5 L each session). Ileal effluent was collected as baseline and once every hour for 8 h after breakfast.

Study Product

The study products were provided in the form of capsules packaged in identical containers in single servings. The DE111® supplement was composed of Bacillus subtilis DE111® (5×10⁹ CFU), medium chain triglycerides, and low-moisture rice maltodextrin. The placebo consisted of an identical capsule containing maltodextrin.

Ileal Effluent Collection and Processing Ileal effluent sample collection was performed on-site by the participants, who emptied their ileostomy pouches into sterile bags (Buerkle™ SteriBag™ StandUp Polyethylene Sampling Bags). After sample collection, the participants placed the samples in a polystyrene box with frozen (−80° C.) cooling packs prior to on-site processing. Samples were collected every hour for 8 h. Upon collection each sample was weighed, diluted 50:50 (w/w) with phosphate buffered saline (PBS) (7.2-7.4 pH) and thoroughly homogenized by vigorous shaking. Aliquots for bacterial enumeration were stored at—80° C. in 40% (v/v) glycerol.

Dry Weight Determination

Samples (1-7 g) were placed in a sterile 20 ml universal container and placed in an oven at 60° C. for 48 h to obtain effluent dry weight. All counts of CFU/g represent the g dry weight of the effluent.

Enumeration of Bacillus Subtilis

Mannitol egg yolk polymyxin agar (MYP, Merck) was used as selective medium for detection of the B. subtilis DE111 ® probiotic (1). Polymyxin B and egg yolk supplements (Merck) were added as recommended by the manufacturer. Colonies of B. subtilis DE111® were identified based on morphology, mannitol fermentation (yellow colored colonies and surrounding area), and absence of lecithinase activity (lack of white halo around the colonies). This identification was confirmed by 16S sequencing of random colonies isolated from several participants using primers 63f-5′-CAGGCCTAACACATGCAAGTC-3′ and 1387r-5′-GGGCGGWGTGTACAAGGC-3. Total B. subtilis counts were done by performing serial 10-fold dilutions of each sample in PBS. Samples were plated on MYP plates, incubated for 18 h hours at 37° C. and colonies counted. To obtain spore counts of B. subtilis DE111®, an aliquot of each sample was heat shocked by incubating at 75° C. for 10 min to inactivate all vegetative cells. Using the effluent dry weights, counts are reported as CFU/g effluent. The number of vegetative cells was calculated using equation 1. Average calculations, including standard deviations, were performed using all participant data points.

Results

Baseline Characteristics of Participants

In total, 274 volunteers were screened for eligibility, of whom 13 were randomized as inclusion criteria were met. One participant was lost to follow-up after the screening visit, another participant dropped out after the first study session. The baseline characteristics of the participants who completed the study are presented in FIG. 2.

Present of Bacillus Subtilis DE111 in the Small Intestine

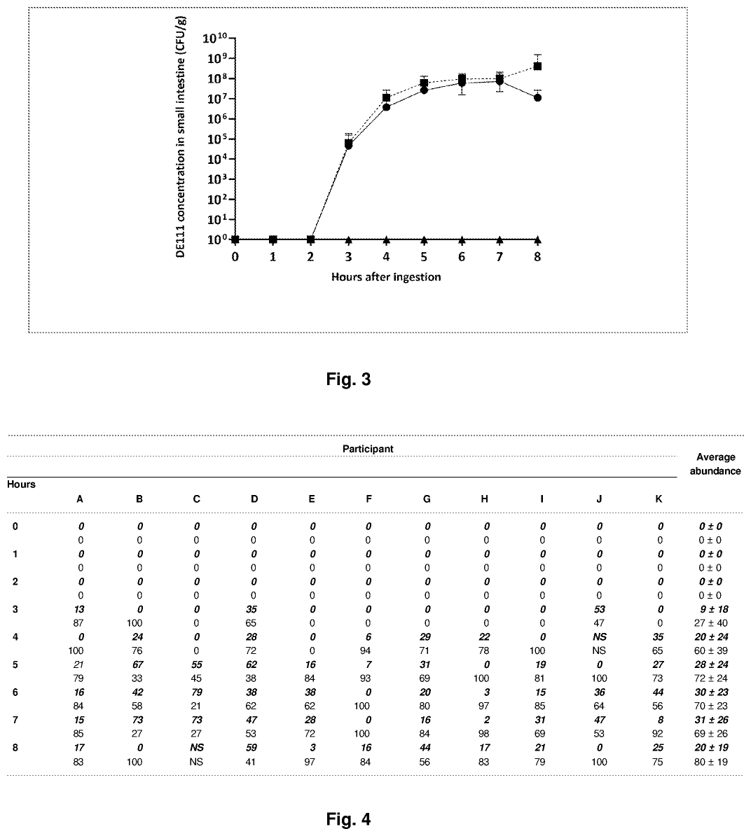

Spores of B. subtilis DE111 ® (6.4×10⁴±1.3×10⁵ CFU/g) were detected in the small intestinal tract 3 h following ingestion of the probiotic capsule (FIG. 3). An increase in the number of spores over time was seen and reached a peak at 6 h following ingestion (9.7×10⁷±8.1×10⁷ CFU/g). The same concentrations of spores continued to be present in the ileal effluent at each time point assessed until end of the study session at 8 h following ingestion. Over the course of the 8-h study session, a total of 3.0×10⁹±6.8×10⁹ CFU of the originally inoculated spores were recovered from the small intestinal effluent. Vegetative cells of B. subtilis DE 111 were also evident after 3 h (4.7×10⁴±1.1×10⁷ CFU/g; FIG. 3), revealing germination of the spore in the small intestine. Vegetative B. subtilis DE111® concentrations in the ileal effluents reached a peak concentration 7 h after ingestion (7.3×10⁷±1.4×10⁸ CFU/g), with the final concentration of 1.2×10⁷±1.4×10⁷ CFU/g at the final time point. All participants had both spores and vegetative cells present in their ileal effluent although the rate at which they first presented and persisted varied among individuals (FIG. 4). Presence of spores and vegetative cells was seen from 3 h after ingestion, with spores identified in 36% of participants and vegetative cells in 27% of samples at this point (FIG. 4). Four hours following ingestion, 80% of participants had spores in their ileal effluents and 60% had vegetative cells. All participant samples had spores present 5 h after ingestion and spores remained present in the effluents until the end of the 8-hour study session (FIG. 4). Detection of vegetative B. subtilis DE 111 ® in ileum effluents was 82% after 5 h, 91% at 6 h, and remained similar until the end of the study. All participants had vegetative cells present in their ileal effluent at some time throughout the session (FIG. 2).

### REFERENCES

(1) Ozkan, A. D., Han, D., Umu, O. C. O., Angun, P., Senturk, B., Yasa, O., et al. (2013). Screening and selection of novel animal probiotics isolated from bovine chyme. Ann. Microbiol. 63, 1291-1300. doi: 10.1007/s13213-012-0588-3.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

What is claimed is:

1. A method for counting Bacillus bacteria directly introduced into the gastrointestinal tract of a human having an ileostomy bag, downstream of the stomach, comprising the steps of:
(a) providing a Bacillus-containing composition comprising one or more Bacillus spp;
(b) administering the Bacillus-containing composition to the human; and
(c) counting the amount of Bacillus bacteria in colony forming units (CFUs).

2. The method of claim 1, wherein the amount of Bacillus bacteria is counted directly from the ileostomy bag.

3. The method of claim 1, wherein the Bacillus-containing composition includes Bacillus subtilis subspecies inaquosorum having accession number NRRL B-67989.

4. The method of claim 1, wherein the Bacillus-containing composition includes 1×10⁶ or more colony forming units (CFUs).

5. The method of claim 1, wherein the Bacillus-containing composition includes about 0.1-10% by weight probiotic and about 90-99.9% by weight standardizing agents.

6. The method of claim 1, wherein the Bacillus-containing composition further comprises an oligosaccharide.

7. The method of claim 1, wherein the Bacillus-containing composition is contained within a capsule or tablet.

8. A method of introducing into the intestinal tract of a human, downstream of the stomach, a beneficial substance, by utilizing a delivery tube having a first end upstream of the stomach and a second end in the gastrointestinal tract downstream of the stomach, the method comprising the steps of:
(a) introducing the beneficial substance into the first end of the delivery tube; and
(b) allowing the beneficial substance to exit the second end of the delivery tube where it enters the intestinal tract downstream of the stomach.

9. The method of claim 8, wherein the beneficial substance is a prebiotic, a probiotic, or a mixture thereof.

10. The method of claim 8, wherein the second end of the delivery tube is in the jejunum.

11. The method of claim 8, wherein the first end of the delivery tube is outside of the nose.

12. The method of claim 8, wherein the delivery tube is an enteral tube feeding device.

13. The method of claim 8, wherein an enteral food is also introduced into the first end of the delivery tube and allowed to exit the second end of the delivery tube.

14. The method of claim 8, wherein the beneficial substance is a prebiotic selected from one or more of a non-starch polysaccharide, a mucopolysaccharide, an oligosaccharide, a fructooligosaccharide (FOS), inulin, chitin, carrageenan, and an AG.

15. The method of claim 8, wherein the beneficial substance is a probiotic selected from one or more of a Bacillus, a Bifidobacterium, and a Bacteroides.

16. The method of claim 15, wherein the Bacillus is selected from the group consisting of B. adolescentis and B. subtilis.

17. The method of claim 16, wherein the Bacillus is Bacillus subtilis subspecies inaquosorum having accession number NRRL B-67989.

18. The method of claim 9, wherein the probiotic includes 1×10⁶ or more colony forming units (CFUs) introduced into the first end of the delivery tube.

19. The method of claim 9, wherein either of the prebiotic or the probiotic has a particle size of 25 microns or less.

20. The method of claim 9, wherein the beneficial substance comprises about 0.1-10% probiotic and about 90-99.9% prebiotic.

21. The method of claim 9, wherein the beneficial substance comprises about 1-10% probiotic and about 90-99% prebiotic.

22. The method of claim 9, wherein the beneficial substance comprises about 0.1-99% probiotic and about 0.1-99.9% prebiotic.
